(19) United States
(12) Patent Application Publication
(10) Pub. No.: US 2004/0247699 A1
(43) Pub. Date: Dec. 9, 2004

(54) Method for the prevention or treatment of overweight in mammals

(76) Inventors: Bernardina Johanna Martina Delsing, S-Hertogenbosch (NL); Gerrit Jan Wiite, Wageningen (NL); Rene John Raggers, Amsterdam (NL); Ladislaus Maria Broersen, Utrecht (NL)

Correspondence Address: YOUNG & THOMPSON, 745 SOUTH 23RD STREET, 2ND FLOOR, ARLINGTON, VA, 22202, US

(21) Appl. No.: 10/323,826

(22) Filed: Dec. 20, 2002

(51) Int. Cl. A61K 35/78

(52) U.S. Cl. 424/725

(57) ABSTRACT

There is provided a method for the prevention or treatment of overweight. More particularly, the invention relates to a method for the treatment or prevention of overweight in a mammal wherein the method comprises the enteral administration of Bacopa plant material to said mammal in an amount effective to reduce the mammal's overweight or to prevent the occurrence of overweight.

### FIELD OF THE INVENTION

The invention relates to a method for the prevention or treatment of overweight, the method comprising the enteral administration of Bacopa plant material.

### BACKGROUND AND OBJECTS OF THE INVENTION

Obesity is very common in nowadays society. Approximately 25% to 35% of the population of the Western world is overweight. Overweight is associated with considerable morbidity and mortality. Obesity is the second preventable death cause in the US and a major risk factor for coronary heart disease, hypertension and diabetes mellitus type II. For obese people it has been shown that a reduction of body weight by 10% decreases the risk for coronary heart disease by 20%. Besides this, overweight and/or excess body fat is generally considered a problem, influencing social behavior and perception of health.

Obesity or overweight is a complex disease that can be treated with various strategies, which are distinguished in the art as given for example in Bray et al, Nature 2000 404(6778):672-7. Bray et al describes several strategies that might lead to significant weight loss, the most important strategies being:

I. Blocking nutrient absorption.

II. Reducing food intake

III. Increasing thermogenesis

IV. Modulating fat or protein metabolism

V. Modulating the central controller regulating body weight.

Each strategy has its own biochemical pathways that can be modulated to achieve the desired weight reducing effects. For example, blockage of nutrient absorption may be achieved by reducing gastrointesinal enzyme activity or blocking gastrointestinal nutrient transporters. Reduction of food intake may be accomplished by modulating centrally acting monoamine and neuropeptide systems, such as inhibiting re-uptake of brain neurotransmitters (e.g. serotonin), influencing adipocyte derived cytokins (e.g. leptin), neuropeptide antagonists, etc.

A typical human has approximately 10 kg of adipose tissue that serves a variety of functions including energy storage in the form of fat and insulation. In obese persons the amount of fat can be in excess of 100 kg. The accumulation of lipid in the fat cells is due to the highly efficient nature of lipogenesis in the adipocyte in response to excess energy intake.

A recognized therapeutic possibility for reducing body fat in e.g. obese or overweight mammals, involves increasing thermogenesis, i.e. the conversion of fat or fatty acids into energy.

Several ingredients capable of stimulating thermogenesis are known in the art e.g. caffeine and ephedrine. However, a main disadvantage of these ingredients is that they stimulate physical activity. This results in restlessness and the inability to sleep (Yates N Z Med J 2000 Jul 28;113(1114):315-7). Hence, ingestion of these ingredients prior to a period of rest, e.g. in the evening or shortly before sleep is intended to commence, is undesirable. Still, there is a need among consumers for an ingredient that is capable of stimulating thermogenesis, e.g. through increasing energy expenditure, stimulating lipolysis and/or decreasing the respiratory quotient (RQ), without increasing physical activity.

The search for such an ingredient has been proven to be extremely cumbersome. Possible ways of searching fox an active ingredient starts with testing ingredients in a cell based on in vitro lipolysis assay. After testing a variety of ingredients, some ingredients may be found to stimulate in vitro lypolysis. However, the capability of stimulating in vitro lypolysis does not ensure any in vivo effect. In fact, only a minority, if any, will have a desired effect in vivo, e.g. increase thermogenesis after oral administration to the mammal. In the rare event that an increased energy expenditure or decreased respiratory quotient (RQ) is observed after oral administration of the ingredient, the probability that side effects such as sleepless or increased activity are absent is minimal. According to applicant's best knowledge, the art has not reported a plant-derived ingredient that increases energy expenditure, without side effects such as increased activity or sleeplessness.

It has now surprisingly been found that Bacopa plant material is capable of increasing thermogenesis in vivo without a simultaneous increase of physical activity, when enterally administered to mammals prior to a period of sleep. Hence, the present invention fulfills a long felt need, by providing a method for stimulating weight reduction, particularly through stimulating thermogenesis, e.g. through stimulating energy expenditure (SE), stimulating lipolysis and/or decreasing the respiratory quotient (RQ)).

Rai Kumar in WO 02/0339 describes the use of a dammarane-type triterpenoid saponin (e.g. bacopasaponim) or derivative or pharmaceutically acceptable salt thereof for treating or preventing conditions, which are related to reduced nitric oxide levels, or which are ameliorable or preventable by augmentation of nitric oxide levels, within the human body, or for promoting responses requiring enhanced nitric oxide levels within the human body. It further describes a method for treating and preventing a condition, which is associated with reduced nitric oxide levels, including Alzheimer's disease; sickle cell disease, sickle cell trait; vascular associated conditions; hypertension and angina; involuntary muscle movement (spasm) or muscle cramps; hepatic conditions; inflammatory conditions (e. g. chilblains); kidney disorders; diabetes (e. g., diabetes mellitus); obesity; migraine and migraine syndrome; drug addiction (e. g., tobacco, marijuana, alcohol etc.); normal pregnancy; preeclampsia, spontaneous preterm labour; aging; menopause associated symptoms (e.g. hot flushes); congenital defects (e.g. limb reduction defects); piles; psoriasis; attention deficit hyperactivity disorder (ADHD); fatigue-related disorders (e.g. obronic fatigue syndrome); hair loss (e.g. male-pattern baldness, age-related baldness); respiratory distress (e.g. pulmonary congestion); female infertility, male infertility; male sexual dysfunction; drug addiction; Helicobacter associated inflammation of the gastric mucosa; AIDS; travel sickness; and osteoclastic bone resorption.

Raj Kumar describes a method for the treatment and prevention of unrelated diseases with the use of a single ingredient. It appears highly unlikely that bacopasaponins will treat or prevent each and every of these diseases. Hence, Raj Kumar erroneously describes obesity as a condition, which is associated with reduced nitric oxide levels. In fact, obesity leads to increased NO production in humans. Choi et al (Clin Chem 2001 Jun;47(6):1106-9) demonstrated that serum NOₓ concentrations were 4.1- and 4.2 fold higher in overweight male and female subjects (BMI≧25.0 kg/m²), respectively, than in underweight subjects (BMI<19.0 kg/m²). Moreover, it has been demonstrated on several occasions that the inhibition of NO release in subcutaneous adipose tissue, increased lipolysis in vivo (Andersson et al, Br I Pharmacol 1999 Apr;126(7):1639-45 and). Elizande et al (J Lipid Res 2000 Aug;41(8):1244-51) describes that studies have shown evidence of inhibition of lipolysis by NO. Hence, absent any further evidence in WO 02/0339, the description to prevent or treat obesity by enhancing NO production would be viewed as erroneous.

WO 99/40897 describes the topical application of Bacopa.

Furthermore, Bacopa plant material has been incorporated in nutritional supplements for its memory enhancing effects (e.g. Bacopa Vitality™) or to improve the growth of new neurons and brains cells (Pinnacle Alpha Dopa Growth Poppers™), Pinnacle Alpha Dopa Growth Poppers™ aims to stimulate muscle growth through the effects of L-dihydroxyphenylalanine (L-Dopa), whereby Bacopa has a supportive role as helping to build new neurons.

### SUMMARY OF THE INVENTION

The present invention provides a method for the treatment or prevention of overweight in a mammal, said method comprising enterally administering to the mammal an effective amount of a Bacopa plant material.

A particular aspect of the present invention provides a method for the treatment or prevention of overweight in a mammal, said method comprising enterally administering to the mammal an effective amount of a Bacopa plant material less than 3 hours before a period of sleep.

In a further aspect the present invention provides a method for stimulating thermogenisis in a mammal, said method comprising enterally administering to the mammal a thermogenic amount of Bacopa plant material, preferably less than 3 hours before a period of sleep.

### BRIEF DESCRIPTION OF THE DRAWINGS

FIG. 1 represents tie results of the experiments performed in Example 1 and illustrates the in vivo effects of Bacopa plant material on energy expenditure.

FIG. 2 represents the results of the experiments performed in Example 2 and illustrates the effects of Bacopa on physical activity.

FIG. 3 represents the results of the experiments performed in Example 3 and illustrates the effects of caffeine on physical activity.

FIG. 4 represents the results of the experiments performed in Example 4 and illustrates the effects of Bacopa plant material on body weight.

### DETAILED DESCRIPTION OF THE INVENTION

The Features of the Present Invention will Hereinafter be Described in Detail.

Bacopa Plant Material

The term Bacopa plant material refers to any plant material obtained from one or more plants belonging to the genus Bacopa, including material obtained by extracting, commuting, drying or other chemical or physical treatment of Bacopa plants or parts thereof. Preferably the Bacopa plant material of the present invention is selected from the group consisting of Bacopa caroliniana, Bacopa egensis, Bacopa eisenii, Bacopa innominata, Bacopa monnieri, Bacopa procumbens, Bacopa repens, Bacopa rotundifolia, Bacopa stricta and mixtures thereof. Bacopa monnieri is most preferably used in the present method. The Bacopa plant material used in accordance with the present invention may be obtained from whole plants or from one or more parts thereof, for example stems, stalks, roots, shoots, rhizomes, tubers, fruits (including seeds), foliage, kernels, husks, hulls or mixtures thereof. Preferably the leaves of the Bacopa plant are used.

According to a preferred embodiment the Bacopa plant material is administered in the form of a plant isolate, more preferably in the form of a plant extract. The term “isolate” as referred to herein, encompasses any fraction that can be obtained from a plant material by means of isolation techniques known in the art, particularly one selected from extraction, distillation or squeezing, and that displays the desired functional properties described herein before. The term “extract” as used in the present invention refers to an isolate that has been obtained by means of solvent extraction. In the preparation of the isolate, the whole plant or a part thereof is preferably first subjected to physical processes prior to the preparation of the isolate. The plants or plant parts are typically subjected to one or more of the following processes prior to the isolation step: grinding, flaking, freezing, drying, commuting and the like. The Bacopa plant or plant parts are thereafter, preferably subjected to one or more isolation processes selected from the group consisting of solvent extraction, cold pressing, hot pressing, distillation, chromatography and filtering, more preferably the Bacopa plant material is subjected to solvent extraction (including supercritical extraction and percolation).

Preferably the solvents used to prepare the Bacopa extract are polar solvents, more preferably a solvent selected from the group consisting of alcohols, water and mixtures thereof, most preferably selected from the group consisting of water, ethanol and mixtures thereof.

Bacopa Fraction with Increased Capacity to Stimulate Thermogenesis

According to a preferred embodiment, the thermogenesis stimulating activity of the isolate of Bacopa plant material used in the present invention is significantly increased by concentrating the thermogenesis stimulating component(s) during the isolation procedure.

Preferably the above is accomplished by fractionating a parent isolate of Bacopa plant material and selecting the fraction(s) showing a significantly increased lipolysis stimulating activity compared to the other fraction(s), for further use.

The fraction(s) of Bacopa isolate so obtained have an increased lipolysis stimulating effect per unit of dry weight compared to the isolate. Preferably these fraction(s) have a lipolysis stimulating capacity per unit of dry weight which is at least twice as high as that of the parent isolate, more preferably at least five times as high, most preferably at least 10 times as high.

Bacosides

Without wishing to by bound by any theory, it is the inventors' belief that the active principle(s) of the Bacopa plant material that provide for the increased thermogenesis are bacosides. Hence, in a preferred embodiment, the present method comprises the administration of an effective amount of bacosides, said bacoside being preferably selected from the group consisting of bacoside A, bacoside B and mixtures thereof. Preferably the bacosides are provided by a Bacopa plant material enriched in bacosides.

Preferably the Bacopa plant material used in the present method comprises at least 1 wt. %, more preferably at least 5 wt. %, even more preferably at least 10 wt. %, even more preferably at least 25 wt. %, most preferably at least 40 wt. % bacosides based on the total dry weight of the Bacopa plant material.

Dosage Bacopa Plant Material

The appropriate daily amount of Bacopa plant material is dependent on the plant species used and/or the type plant material used (e.g. fresh or dried commuted plant material, extract or fraction). Preferably, a thermogenically effective amount of Bacopa plant material is administered in the present method.

By thermogenically effective amount it is meant that the Bacopa plant material, particularly the Bacopa plant extract, is administered in an amount, preferably a daily amount, equivalent to between 0.01 and 250 mg dry Bacopa plant material per kg of body weight, more preferably between 1 and 100 mg/kg, even more preferably between 2 and 75 mg/kg, most preferably between 3 and 50 mg/kg.

In a further preferred embodiment of the present method, bacosides are administered, preferably through the Bacopa plant material, in a daily amount of between 0.01 and 250 mg bacoside per kg of body weight, more preferably between 0.5 and 100 mg/kg, even more preferably between 1 and 75 mg/kg, most preferably between 2 and 50 mg/kg.

Preferably the Bacopa plant material is administered in a dosage. Whenever the term dose or dosage is used within this disclosure, any dosage form is encompassed which can be a administered enterally, within a fairly narrow time span such as pills, tablets and capsules. Whenever reference is made to a certain quantity that is administered per dose or dosage, said quantity is preferably administered within a period of one hour, more preferably within 15 minutes, even more preferably within 5 minutes.

In a further aspect, the present method preferably comprises the administration of a dosage containing between 0.01 and 250 mg Bacopa plant material per kg of body weight, more preferably between 0.5 and 100 mg/kg, even more preferably between 1 and 50 mg/kg. For a human, the present method preferably comprises the administration of a dosage containing between 30 mg and 5 grams, more preferably between 50 mg and 4 grams, even more preferably between 100 mg and 2.5 grams, most preferably between 250 mg and 2 grams Bacopa plant material.

Additional components, as described hereinafter that are not detrimental to the effect of Bacopa can be added.

Stimulating Growth

In a preferred embodiment the present method does not comprise the coadministration of tissue growth enhancing substances, since these might increase bodyweight. For example, L-dihydroxyphlenylalanie (L-Dopa) has been suggested to stimulate muscle growth. Hence, preferably the present method comprises the administration of a dosage being substantially free, of L-Dopa, preferably a, dosage being substantially free of muscle growth stimulating ingredients, more preferably a dosage being substantially free of tissue growth simulating ingredients.

Treatment and Prevention of Overweight

The present invention provides a method for the prophylactic and/or curative treatment of overweight and obesity, more preferably a method for decreasing the size and growth of adipose tissue mass. In a further preferred embodiment, the present invention provides a method for stimulating thermogenesis, preferably a method for increasing energy expenditure.

The term “overweight” as used in the present invention refers to a bodyweight that is above the desired bodyweight of a human subject or that of a pet or farm animal as defined by its owner.

The term “stimulating thermogenesis” as use in the present invention refers to the process of increasing energy expenditure (RE) and/or stimulating lipolysis and/or decreasing the respiratory quotient (RQ).

The term “energy expenditure” as used in the present invention refers to the metabolic rate of a mammal, and the term “respiratory quotient” refers to the ratio carbon dioxide production: oxygen consumption. The RQ values are close to 0.7 for fat oxidation and 1.0 for carbohydrate oxidation.

The present method is advantageously used by humans, in particular human subjects who have abody mass index above 25. The method can also be advantageously used in animals selected from the group of dogs and cats. Additionally the method can be used to increase lean body mass, in particular in farm animals, preferably selected from the group of cattle, pigs, horses, sheep and goats.

In a particular preferred embodiment the Bacopa plant material is incorporated in a nutritional supplement or dietary nutritional product for use in a method aimed to reduce overweight, and/or reduce adipose tissue mass, and/or stimulate thermogenesis. Packaged products, which have been provided with labels that explicitly or implicitly direct the consumer towards the use of said supplement or product in accordance with one or more of the above or below purposes, are encompassed by the present invention. Such labels may for example make reference to the method for the treatment of overweight by incorporation of terminology like “slim”, “lean”, “weight reduction”, “light”, “diet”, “fat-burner” and the like. The overweight reducing properties of the product may be indicated via indicia such as pictures, drawings and other indicia, from which a consumer can conclude that the product aims to treat or prevent overweight or stimulate thermogenesis.

Administration

The Bacopa plant material was surprisingly found to lack a physical activity stimulating effect, thereby making it particularly suitable for administration before or during a period of rest. Thus, preferably, the present invention provides method for stimulating thermogenesis in a mammal said method comprising enterally administering to the mammal a thermogenic amount of Bacopa plant material. More preferably, the present method comprises stimulating thermogenesis in a resting mammal, most preferably in a sleeping mammal.

The term “resting mammal” as used in the present invention preferably refers to a mammal that does not or only minimally perform coordinated movements. The term “sleep” is defined as the state of rest characterized by relative physical and nervous inactivity, lessened responsiveness and unconsciousness.

In the present method, the Bacopa plant material is preferably ingested less than 3 hours more preferably less than 2 hours, even more preferably before less than 1 hour before a period sleep.

In a preferred embodiment the present method comprises the administration of the Bacopa plant material within a period staring 30 minutes after dinner of one day and ending 30 minutes before breakfast the next day, more preferably at any time within the period starting after dinner and ending when sleep commences.

In a further preferred embodiment the Bacopa plant material is administered between 7 PM of one day and 7 AM the next day, more preferably between 8 PM and 12 PM of the same day, even more preferably between 9 PM and 12 PM of the same day.

Packaged products, which have been provided with labels that explicitly or implicitly direct the consumer towards the use of said supplement within a period indicated above, are encompassed by the present invention. Such labels may for example suggest the, intended period for administration by incorporation of terminology like “sleep” “night-time”, “evening”, “bedtime” and the like. The intended period for administration of the product may also be indicated via indicia such as pictures, drawings (e.g. a clock or a moon) and other indicia from which a consumer can conclude that the product is intended for administration within the above identified period.

The Bacopa plant material is administered enterally, in particular orally, preferably swallowed immediately after application to the oral cavity.

Many weight-reducing components lose their weight-reducing efficacy within a short period, due to adaptation of the body. In contrast, it has been found that Bacopa plant material is deemed to be effective over a long period of time. Hence, in a further preferred embodiment the present method comprises the administration of Bacopa plant material for a period of at least 3 consecutive days, preferably for at least 5 consecutive day.

The plant material used in the present method can be applied in any suitable form for its administration, such as meals, bars, pills, capsules, gels, biscuits and drinks. According to a preferred embodiment the Bacopa plant material is provided in a unit dosage form.

The term “unit dosage form” refers to a physically discrete unit suitable for unitary administration to human subjects and other mammals, wherein each unit contains a predetermined quantity of Bacopa plant material and a pharmaceutically acceptable carrier. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

The aforementioned unit dosage form is preferably in a solid or semisolid form, more preferably in the form of an oral dosage unit, which term includes capsules, tablets, microparticles and microspheres. The solid or semisolid unit dosage form preferably has a weight between 0.1 and 30 grams, more preferably between 0.2 and 10 gram. When an oral dosage unit is used to provide the Bacopa plant material, it preferably has a weight between 0.2 and 4 grams, even more preferably between 0.5 and 3 grams. In the present method a (daily) dosage of Bacopa plant material can include one or more unit dosage forms, preferably the daily dosage consists of 1 to 3 unit dosage forms.

Combinations

It was found that the Bacopa plant material can be advantageously combined with a plant material selected from the group consisting of hops preferably Humulus lupulus), chamomile (preferably Matricaria chamomillae), lemon balm (preferably Melissa officinalis), passionflower (preferably Passiflora incarnata), Scutellaria (preferably Scutellaria baicalensis) and valerian (preferably Valeriana officinalis). These plant materials are preferably administered in an amount of with between 0.01 and 250 mg plant material per kg of body weighs more preferably between 0.5 and 100 mg/kg, even more preferably between 1 and 50 mg/kg. For a human, the present method preferably comprises the administration of a dosage containing Bacopa plant material and between 30 mg and 5 grams, more preferably between 50 mg and 4 grams, even more preferably between 100 mg and 2.5 grams, most preferably between 250 mg and 2 grams of a plant material selected from the group consisting of hops (preferably Humulus lupulus), chamomile (preferably Matricaria chamomillae), lemon balm (preferably Melissa officinalis), passionflower preferably Passiflora incarnata), Scutellaria (preferably Scutellaria baicalensis) and valerian preferably Valeriana officinalis).

The combined preparation further improves sleep quality or improves sleep inducing properties, without (or only minimally) effecting the weight reducing properties of Bacopa plant material.

The hops, chamomile, passionflower, melissa or valerian plant material used in accordance with the present invention may be obtained from whole plants or from one or more parts thereof, for example stems, stalks, roots, shoots, rhizomes, tubers, fruits (including seeds), foliage, kernels, husks, hulls or mixtures thereof.

According to a preferred embodiment the material to be combined is administered in the form of a plant isolate, more preferably in the form of a plant extract. The term “isolate” as referred to herein, encompasses any fraction that can be obtained from a plant material by means of isolation techniques known in the art, particularly one selected from extraction, distillation or squeezing, and that displays the desired functional properties described herein before. The term “extract” as used in the present invention refers to an isolate that has been obtained by means of solvent extraction. In the preparation of the isolate, the whole plant or a part thereof is preferably first subjected to physical processes prior to the preparation of the isolate. The plants or plant parts to be combined are typically subjected to one or more of the following processes prior to the isolation step: grinding, flaking, freezing, drying, commuting and the like. The plant or plant parts are thereafter preferably subjected to one or more isolation processes selected from the group consisting of solvent extraction, cold pressing, hot pressing, distillation, chromatography and filtering, more preferably the plant material is subjected to solvent extraction (including supercritical extraction and percolation).

Preferably the solvents used to prepare the plant extract are polar solvents, more preferably a solvent selected from the group consisting of alcohols, water and mixtures thereof, most preferably selected from the group consisting of water, ethanol and mixtures thereof.

According to a preferred embodiment valerian plant material is used in the present method, more preferably valerian extract, even more preferably valerian root extract. Preferably the valerian plant material is enrich in valerenic acid or a pharmaceutically acceptable salt thereof. Advantageously the valerian plant material contains at least 0.2 wt. %, more preferably at least 0.5 wt. %, even more preferably at least 0.75 wt. %, most preferably at least 1 wt. % valerenic acid based on the dry weight of the valerian plant material.

According to a further preferred embodiment, Scutellaria plant material is in the present method, preferably Scutellaria baicalensis. Preferably the Scutellaria plant material is provided as an extract.

Preferably, the Bacopa plant material and plant material to be combined administered in a unit dosage form, wherein the weight ratio Bacopa plant material: plant material to be combined is between 100:1 and 1:1, preferably between 10:1 and 1:1.

The combination of melatonin and Bacopa plant material are also envisaged for the present invention. The Bacopa plant material may also be advantageously combined with melatonin, to improve sleep quality and/or improve sleep inducing properties. Preferably the present method comprises coadministration of Bacopa plant material and melatonin. Preferably the present method comprises the administration of a dosage containing Bacopa plant material and less than 10 mg melatonin. Preferably, the dosage administered contains between 0.01 and 5.0 mg, more preferably between 0.1 and 2.5 mg of melatonin.

Of course, combination of Bacopa plant material, melatonin and a plant material as defined hereinbefore are also envisaged for use herein.

The present invention will hereinafter be described more specifically in the following non-limiting Examples and Comparative Examples

### EXAMPLE 1

In vivo Effects of Bacopa Plant Material on Energy Expenditure

The effects of Bacopa monnieri on energy expenditure was tested in rats. In a placebo controlled cross-over design Bacopa monnieri was tested in 4 months old male Wistar rats (n=6). The rats were each placed in a separate metabolic cage (Oxyax Equal Flow System, Colmbus Instruments, Ohio, USA) at 8.30 am. (lights on at 7.00 a.m.) and basal metabolism was measured during 1½ hour. At 10.00 am., the animals were shortly removed from the metabolic cage for administration of the test compound. Rats are normally asleep at that time of day. The concentration of oxygen and carbon dioxide in each cage was measured every 26 minutes.

Following the determination of basal metabolism, either placebo (1 ml water) or 100 mg Bacopa monnieri extract per kg of body weight dissolved in 1 ml water (Bacomax 40™, Renaissance Herbs) was administered via oral gavage. Subsequently, the effects on O₂ consumption (VO₂) and CO₂ production (VCO₂) were monitored during 5 hours. Energy expenditure was calculated with the following formula:

EE=(3.815+1.232×VCO₂/VO₂)*VO₂

wherein: VO₂=oxygen consumption; and

VCO₂=carbon dioxide production.

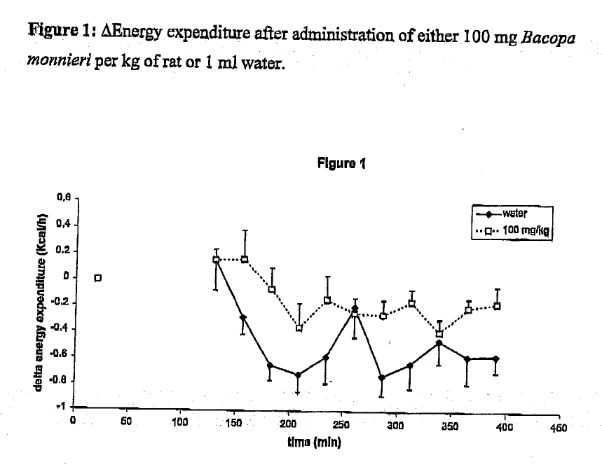

FIG. 1 shows the average difference in energy expenditure of rats that received either placebo or Bacopa monnieri with to basal energy expenditure. FIG. 1 shows an increased energy expenditure in rats that received 100 mg Bacopa monnieri per kg of rat compared to placebo in the period when rats are normally resting (sleeping). The results are indicative for the energy expenditure stimulating effects, thermogenesis increasing and weight reducing properties of Bacopa monnieri in resting mammals.

### EXAMPLE 2

Effects of Bacopa on Physical Activity

The effects of Bacopa monnieri on physical activity was tested in rats and compared to placebo (water). In the study as described in example 1, the physical activity of the animals was determined by monitoring ambulatory counts.

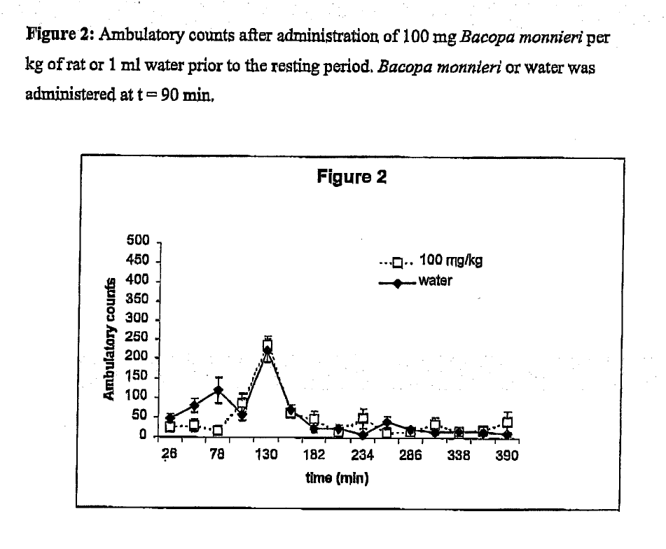

Results are shown as the average ambulatory counts per 26 minutes. FIG. 2 shows that ambulatory counts do not differ between the rats receiving placebo or 100 mg Bacopa monnieri per kg of rat in the period when rats are normally asleep. The results are indicative for lack of side effects such as sleeplessness and restlessness, after oral administration of Bacopa monnieri to resting mammals.

### COMPARATIVE EXAMPLE 3

Effects of Caffeine on Physical Activity

The effects of caffeine on physical activity was tested in rats and compared to placebo (water). In a placebo controlled cross-over design as described in example 1, the effects of administration of either 12.5 mg caffeine per kg of rat or water on physical activity was determined. Physical activity of the animals was determined by monitoring ambulatory counts.

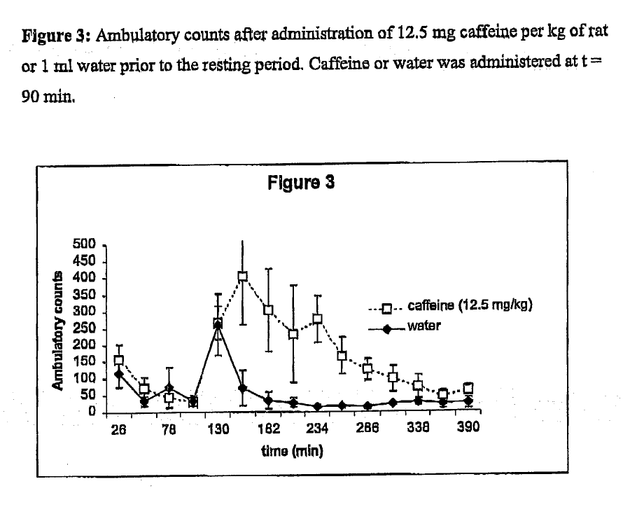

Results are shown as the average ambulatory counts per 26 minutes. FIG. 3 shows that the ambulatory counts increase after rats have received 12.5 mg caffeine per kg rat compared to placebo in the period when rats are normally asleep. The results are indicative for the side effects such as sleeplessness and restlessness, after oral administration of caffeine to resting mammals.

### EXAMPLE 4

Body Weight Reducing Effects of Bacopa monnieri

Twenty-three 10 weeks old male Sprague Dawley rats (Harlan, The Netherlands) were housed in groups of 3-4 rats per cage. Rats were housed at controlled room temperature (21±2° C.) and relative humidity of 60±10% under normal 12 h light-12 h dark cycle conditions (lights on at 07:00). Throughout the experiment, standard rodent food (Special Diet Services, Witham, UK) and tap water were freely available in the home cages.

Upon arrival, rats were allowed to acclimatize in the animal quarter for one week. Then, rats were randomly assigned to one of the treatment groups, in such a way that not more than 2 rats in the same cage received the same treatment. From that moment on, rats received daily oral injections at the start of the light phase, i.e. between 08:00 and 11:00 a.m. (before the sleeping period). Just before each administration, the body weight was determined. Saline, 50 and 500 mg Bacopa extract (Bacopa monnieri extract powder-20%, Indfrag Limited, India) per kg rat were injected orally in a volume of 2 ml/kg. Groups consisted of 8, 7 and 8 rats respectively. Treatment continued for 9 consecutive days.

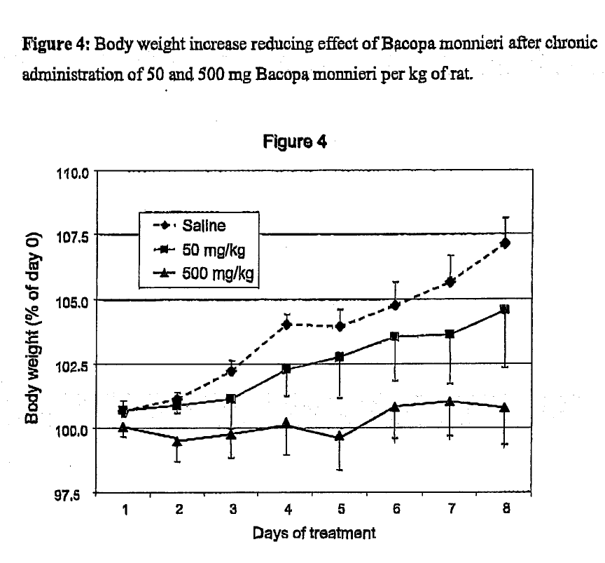

The effects of oral administration of different doses of Bacopa extract on body weight changes in rats are shown in FIG. 4. Data are expressed as percentage of the body weight prior to treatment. Repeated measure ANOVA (using Statistical Package for the Social Sciences (SPSS) for Windows, Release 11.0.1.) with the within-subject factor Dose (having 3 levels) and the between-subject factor Days (having 8 levels) was applied to the data. The analysis revealed a significant main effect of Dose [F(2,20)=3.64, p<0.05], a significant main effect of Days [F(7,140)=13.55, p<0.001], and a significant Dose×Days interaction [F(14,140)=2.11, p<0.02]. This represents a significant reduced body weight increase between groups. Subsequent post-hoc tests indicated that the 500 mg/kg dose significantly decreased body weight relative to saline [p<0.02].

The results are indicative for the body weight reducing effects of Bacopa plant material, particularly when administered orally before a period of sleep.

1. A method for the treatment or prevention of overweight in a mammal, said method comprising enterally administering to the mammal an effective amount of Bacopa plant material.

2. Method according to claim 1, wherein the Bacopa plant material is administered less than 3 hours before a period of sleep.

3. Method according to claim 1, wherein the Bacopa is administered in a dosage, said dosage containing between 30 mg and 5 grams Bacopa plant material.

4. Method according to claim 1, for stimulating thermogenesis in a mammal.

5. Method according to claim 1, wherein the method comprises the administration of a Bacopa plant isolate.

6. Method according to claim 1, wherein the Bacopa is administered in a dosage, said dosage being substantially free of L-dihydroxyphenylalanine.

7. Method according to claim 1, wherein the Bacopa plant material is obtained from Bacopa monnieri.

8. Method according to claim 1, wherein the method comprises the administration of Bacopa plant material in an amount equivalent to between 0.01 and 250 mg dry Bacopa plant material per kg of body weight

9. Method according to claim 1, wherein the method comprises the oral administration of a Bacopa plant material.

10. Method according to claim 1, wherein the Bacopa plant is administered in a unit dosage form, said unit dosage form further containing a pharmaceutically acceptable carrier.

11. Method according to claim 1, wherein the method further comprises the administration of a plant material selected from the group consisting of hops, chamomile, lemon balm, passionflower, Scutellaria and valerian.

12. Method according to claim 1, wherein the method further comprises the administration of valerian plant material.

13. Method according to claim 1, wherein the method further comprises the administration of Scutellaria plant material.

14. Method according to claim 1, wherein the method further comprises the administration of melatonin.
